(19) United States
(12) Patent Application Publication
(10) Pub. No.: US 2003/0211149 A1
(43) Pub. Date: Nov. 13, 2003

(54) Extended release tablets comprising felodipine

(76) Inventor: Bernard Charles Sherman, Toronto (CA)

Correspondence Address: NIXON & VANDERHYE P.C., 8th Floor, 1100 North Glebe Road, Arlington, VA, 22201-4714, US

(21) Appl. No.: 10/139,347

(22) Filed: May 7, 2002

(51) Int. Cl. A61K 35/78; A61K 9/00; A61K 9/22

(52) U.S. Cl. 424/468; 424/731; 514/355

(57) ABSTRACT

An extended-release tablet comprising felodipine, a non-ionic surfactant, and a release-controlling excipient, wherein the amount of surfactant is more than 0.01 part but less than 1.0 part per part felodipine by weight.

### BACKGROUND OF THE INVENTION

Felodipine is a substituted dihydropyridine that is effective as a calcium channel blocker, useful for the treatment of hypertension.

Extended-release tablets comprising felodipine are sold in the United States and elsewhere under the tradename Plendil™ in strengths of 2.5 mg, 5 mg, and 10 mg.

Felodipine has very low solubility in water, which makes it difficult to formulate tablets comprising felodipine that will enable maximum absorption upon oral administration.

Accordingly, tablets for oral administration comprising felodipine require a mechanism to increase the rate and extent of dissolution of the felodipine when it is released from the tablet into the gastro-intestinal fluid. Extended-release tablets comprising felodipine also require another mechanism to extend and control the rate at which the felodipine is released from the tablet.

Mechanisms to enable both adequate dissolution and extended-release are known in the prior art.

U.S. Pat. No. 4,803,081 discloses an extended-release felodipine tablet comprising a solution or a dispersion of felodipine in a semi-solid or liquid non-ionic solubilizer (i.e. surfactant), wherein the amount by weight of the surfactant is at least equal to the weight of felodipine, and further comprising a release controlling agent to provide extended-release. Plendil™ tablets ™—Registered trademark

appear to be made according to the teaching of this patent. In Plendil™ tablets, the non-ionic surfactant is polyoxyl 40 hydrogenated castor oil, and the agent to provide extended-release is a hydrophilic gel-forming polymer, specifically hydroxypropyl methylcellulose.

The formulation of Plendil™ tablets has the disadvantage that the amount of non-ionic surfactant required is relatively large. Since the non-ionic surfactant softens the tablets, it is necessary that the tablets be relatively large, so as to contain enough of other excipients (i.e. inactive ingredients) to overcome the softening effect of the non-ionic surfactant.

A Plendil™ tablet of 10 mg strength has a weight of about 470 mg including the film coating, so that the weight of the core tablet is about 450 mg. The amount of excipients in a core tablet is thus about 44 times the amount of felodipine by weight.

U.S. Pat. No. 6,132,772 discloses a formulation in which the low solubility of a drug such as nifedipine or felodipine is overcome by dissolving it in molten polyethylene glycol having a mean molecular weight of over 1000. No example is given using felodipine specifically. However, due to the relatively low solubility of felodipine in polyethylene glycol, the ratio of polyethylene glycol to felodipine would have to be about 10 to 1 or higher to avoid precipitation of felodipine crystals on long term storage. Also, other excipients have to be added to provide for extended-release, so that extended-release felodipine tablets following the teaching of U.S. Pat. No. 6,132,772 would again be relatively large.

In light of this prior art, an objective of the present invention is to enable felodipine extended-release tablets in which the felodipine is solubilized by a non-ionic surfactant, but in which the amount of surfactant is less than one part per part felodipine.

### BRIEF SUMMARY OF THE INVENTION

The tablets of the present invention are extended-release tablets comprising felodipine, a non-ionic surfactant, and a release-controlling excipient, wherein the amount of surfactant is more than 0.01 part but less than 1.0 part per part felodipine by weight, when made by a process comprising the steps of dissolving the felodipine and surfactant in organic solvent, drying to evaporate the solvent, and further processing the dried material into tablets.

### DETAILED DESCRIPTION OF THE INVENTION

As aforesaid, the tablets of the present invention comprise felodipine and a non-ionic surfactant.

Preferred surfactants are those that are water-soluble and are liquid or semi-solid at 25° C., including, for example:

i) Reaction products of natural or hydrogenated vegetable oils and ethylene glycol; i.e. polyoxyethylene glycolated natural or hydrogenated vegetable oils; for example, polyoxyethylene glycolated natural or hydrogenated castor oils. Especially suitable are the compounds listed in the United States Pharmacopoeia and National Formulary as polyoxyl 35 castor oil, and polyoxyl 40 hydrogenated castor oil. Most preferred is polyoxyl 35 castor oil.

ii) Polyoxyethyl-sorbitan-fatty acid esters; e.g. lauryl, palmityl, stearyl and oleyl esters e.g. of the type known and listed in the United States Pharmacopoeia and National Formulary as polysorbates. Especially suitable products of this class are polysorbate 20 and polysorbate 80.

The amount of surfactant, per part felodipine by weight, will be more than 0.01 part but less than 1.0 part, will preferably be from 0.1 part to 0.5 part, and will more preferably be from 0.1 part to 0.3 part.

In order to achieve an intimate mixture of the surfactant with the felodipine, the process of manufacture of the tablets will include the steps of dissolving the felodipine and surfactant in organic solvent, and evaporating the organic solvent.

The organic solvent will preferably comprise a lower alcohol, such as methanol, or a chlorinated hydrochloride, such as methylene chloride.

Optionally, excipients other than the solubilizer may be dissolved in the organic solvent along with the felodipine and surfactant before the solvent is evaporated. The total of excipients by weight, including the surfactant, that are dissolved in the organic solvent along with the felodipine will preferably be less than the amount of felodipine by weight. However, more preferably there will be no other such excipient, so that the solution will comprise only felodipine and the surfactant dissolved in the solvent.

The evaporation of the solvent will preferably be done such that, upon drying, the felodipine and surfactant will be in amorphous form; that is to say, the felodipine will not be precipitated as crystals.

This may be done, for example, by spray-drying of the solution.

Alternatively, the solution may be sprayed onto excipient, while drying in a fluid-bed dryer. Such other excipient will preferably comprise part or all of release-controlling excipient. Alternatively, the solution may be mixed into another excipient, and the mixture dried to evaporate the solvent. Again, the other excipient will preferably comprise some or all of the release-controlling excipient.

As aforesaid, the tablets will comprise a release-controlling excipient. This excipient will be preferably a gel-forming polymer or gum; that is to say, a polymer or gum that forms a gel when added to water. This excipient will preferably be a hydrophilic cellulose derivative, such as hydroxypropyl cellulose, hydroxyethyl cellulose, hydroxypropyl methylcellulose, or methylcellulose.

Especially preferred is hydroxypropyl methylcellulose, which is available in grades differing in degrees of substitution and molecular weight. Most preferred is hydroxypropyl methylcellulose having 19-24% methoxyl substitution and 7-12% hydroxypropyl substitution. This polymer is sold by Dow Chemical Co. under the tradename Methocel™ in types K100M, K15M, K4M, K100LV and K3. Type K100M is the grade having the highest mean molecular weight and highest solution viscosity, and type K3 has the lowest mean molecular weight and solution viscosity. Methylcellulose is also sold by Dow Chemical Co. under the tradename Methocel™ in types A4M and A15LV. Type A4M has a higher mean molecular weight and solution viscosity than type A15LV.

The tablets of the present invention may contain other excipients in addition to the solubilizer and release-controlling excipient. For example, there may be included a lubricant needed to avoid sticking to the punches in the tabletting process. Such lubricant may be, for example, stearic acid or a stearate, such as magnesium stearate. Similarly, there may be included a glidant, such as colloidal silicon dioxide, to improve flow of the mixture in the tabletting process.

The tablets will be produced by compressing the final mixture of ingredients on a tablet press. To improve flow for the tabletting process the mixture may first be compacted and reground into granules, and then the resulting granules will be recompressed into tablets.

The total amount of excipients in the tablet per part felodipine by weight will preferably be less than 44 parts, more preferably less than 40 parts, even more preferably less than 35 parts, even more preferably less than 30 parts, even more preferably less than 25 parts, even more preferably less than 20 parts and most preferably less than 15 parts.

The tablets may be uncoated or may have a film-coating applied to their surface, using any of various polymer systems and process well known in the art.

The present invention will be further understood from the following examples, which are intended to be illustrative and not limiting the scope of the invention.

### EXAMPLE 1

A granulation comprising 33.33% felodipine by weight was made using ingredients in the following proportions:

| | |
|---|---|
| Felodipine | 100. |
| Polyoxyl 35 Castor Oil | 20. |
| Methylene Chloride | 240. |
| Methocel ™ K100LV | 180. |
| Total Excluding Solvent | 300. |

The felodipine and polyoxyl 35 castor oil were dissolved in the methylene chloride. The solution was then slowly added to the Methocel™ K100LV while mixing in a heated mixer, so that the methylene chloride was continuously evaporated as the solution was being added.

### EXAMPLE 2

Ingredients were mixed in the proportions as follows:

| | |
|---|---|
| Felodipine 33.33% granulation from example 1 | 30.0 |
| Methocel ™ A15LV | 99.4 |
| Stearic Acid | 0.4 |
| Colloidal Silicon Dioxide | 0.2 |
| | 130.0 |

The ingredients were mixed together and the mixture was processed into tablets at a net weight of 130 mg per tablet. Each tablet thus comprised 10 mg of felodipine, 2 mg of polyoxyl 35 castor oil, 18 mg of Methocel™ K100LV, 99.4 mg of Methocel™ A15LV, 0.4 mg of stearic acid, and 0.2 mg of colloidal silicon dioxide.

1. An extended-release tablet comprising felodipine, a non-ionic surfactant, and a release-controlling excipient, wherein the amount of surfactant is more than 0.01 part but less than 1.0 part per part felodipine by weight, when made by a process comprising the steps of dissolving the felodipine and surfactant in organic solvent to form a solution, drying to evaporate the solvent, and further processing the dried material into tablets.

2. A tablet of claim 1 wherein an excipient other than the surfactant is also dissolved in the organic solvent, with the proviso that the total amount of excipients, including the surfactant, that are dissolved in the solvent is less than the amount of felodipine by weight.

3. A tablet of claim 1 wherein no excipient other than the surfactant is dissolved in the organic solvent.

4. A tablet of any of claims 1 to 3 wherein the surfactant is water-soluble and is a liquid or semi-solid at 25° C.

5. A tablet of any of claims 1 to 4 wherein the surfactant is a reaction product of a natural or hydrogenated vegetable oil and ethylene glycol.

6. A tablet of claim 5 wherein the surfactant is polyoxyethylene glycolated natural or hydrogenated castor oil.

7. A tablet of any of claims 1 to 6 wherein the surfactant is a polyoxyethylene-sorbitan-fatty acid ester.

8. A tablet of any of claims 1 to 7 wherein the amount of surfactant is from 0.05 part to 0.5 part per part felodipine by weight.

9. A tablet of claim 8 wherein the amount of surfactant is from 0.1 part to 0.3 part per part felodipine by weight.

10. A tablet of any of claims 1 to 9 wherein the release-controlling excipient is a gel-forming polymer or gum.

11. A tablet of claim 10 wherein the release-controlling excipient is a hydrophilic cellulose derivative.

12. A tablet of claim 11 wherein the release-controlling excipient is selected from the group consisting of hydroxypropyl cellulose, hydroxyethylcellulose, hydroxypropyl methylcellulose or methylcellulose.

13. A tablet of claim 12 wherein the release-controlling excipient is hydroxypropyl methylcellulose.

14. A tablet of any of claims 1 to 13 that comprises both hydroxypropyl methylcellulose and methylcellulose.

15. A tablet of any of claims 1 to 14 wherein the process comprises the steps of dissolving the felodipine and surfactant in organic solvent, adding the solution to another excipient, and evaporating the solvent.

16. A tablet of claim 15 wherein the excipient to which the solution is added comprises part or all of the release-controlling excipient.

17. A tablet of any of claims 1 to 16 wherein the solvent comprises a lower alcohol.

18. A tablet of any of claims 1 to 16 wherein the solvent comprises methanol.

19. A tablet of any of claims 1 to 18 wherein the solvent comprises a chlorinated hydrocarbon.

20. A tablet of claim 18 wherein the solvent comprises methylene chloride.

21. A tablet of any of claims 1 to 20 wherein the total of excipients is less than 44 parts per part felodipine by weight.

22. A tablet of claim 21 wherein the total of excipients is less than 40 parts per part felodipine by weight.

23. A tablet of claim 22 wherein the total of excipients is less than 35 parts per part felodipine by weight.

24. A tablet of claim 23 wherein the total of excipients is less than 30 parts per part felodipine by weight.

25. A tablet of claim 24 wherein the total of excipients is less than 25 parts per part felodipine by weight.

26. A tablet of claim 25 wherein the total of excipients is less than 20 parts per part felodipine by weight.

27. A tablet of claim 26 wherein the total of excipients is less than 15 parts per part felodipine by weight.
